(19) United States
(12) Patent Application Publication
(10) Pub. No.: US 2017/0319177 A1
(43) Pub. Date: Nov. 9, 2017

(54) ULTRASOUND PROBE HAVING PUNCTURE GUIDING FUNCTION

(71) Applicants: Hui-Hua CHIANG, Taipei (TW); Chien-Kun TING, Taipei City (TW); Broadsound Corporation, Jhubei City, Hsinchu County (TW)

(72) Inventors: Hui-Hua CHIANG, Taipei (TW); Chien-Kun TING, Taipei City (TW); Ying-wei YUAN, Jhubei City, Hsinchu County (TW)

(21) Appl. No.: 15/534,992

(22) PCT Filed: Dec. 12, 2014
(86) PCT No.: PCT/CN2014/093683

§ 371 Date: Jun. 9, 2017

(51) Int. Cl. A61B 8/00; A61B 8/08; A61B 17/34; A61B 34/20; A61B 17/17

(52) U.S. Cl. CPC A61B 8/4455; A61B 17/1725; A61B 8/0841; A61B 17/3403; A61B 17/1728; A61B 34/20; A61B 17/1703; A61B 2034/2063; A61B 2017/3413

(57) ABSTRACT

An improved ultrasound probe (100) has an ultrasound transducer as a body (10) and is capable of accurate positioning on target operative regions of a patient after being improved. The ultrasound probe (100) at least comprises the body (10) and an engagement member (20); the body (10), at an end thereof, comes into contact with patient skin (S), and is provide, on a side surface thereof, with a groove (11) extending from the end that can contact the patient skin (S); the engagement member adjoins the groove (11) of the body (10), allowing one side surface of an article to be disengaged from or engaged in the groove (11), and the engagement member (20) is located at a first location and a second location relative to the groove (11); the article is engaged in the groove (11) when the engagement member (20) is located at the first location relative to the groove (11), and on the contrary, the article is disengaged from the side surface of the body (10) when the engagement member (20) is located at the second location relative to the groove (11).

### RELATED APPLICATIONS

This is a National Phase Application filed under 35 U.S.C. 371 as a national stage of PCT/CN2014/093683 filed Dec. 12, 2014, the content of which is hereby incorporated by reference in its entirety.

### BACKGROUND

### Technical Field

The present invention relates to an ultrasound probe. More particularly, the present invention discloses an ultrasound probe with a groove for allowing an article to be engaged into or detached from the groove through one side surface thereon.

### Description of Related Art

In clinical surgeries, it usually needs puncturing performing by a needle. However, in part of portions, puncturing will be difficult and accompanied with risks due to the complex structure. Such as taking anesthesia, a para-anesthesia includes spinal anesthesia and epidural anesthesia. Wherein the epidural anesthesia can be applied on labor analgesia or patient-controlled analgesia, has a higher technical difficulty.

Epidural anesthesia is used to inject a local anesthetic into epidural space for temporarily blocking the neural network. The above operation is quite dependent on experience of an operator, such as anesthesiologists. When a puncture needle is punctured into the target area from the back, it must be accurately punctured into the epidural space with a width of only 2 mm-7 mm after the path of a blind puncture. Furthermore, inserting a catheter into the epidural space along the puncture path of the puncture needle, then removing the puncture needle. The anesthetic will be injected through the catheter. However, there is still lacking a clear and objective method for determining the position where the puncture needle arrives. Thus, there is a clinical risk of over puncturing so as to result in failures and complications, such as post dural puncture headache.

As mentioned above, the traditional method dependents on the experience of the operator, so that the risk of anesthesia for patients will increase. In order to avoid the puncture failure, many positioning technologies have been developed, such as pressure, electric or optical methods. However, there is still no visualizable information for anesthesia in clinical. Although, in some of case, B-mode ultrasound is used for exosomatic guiding the puncture needle to puncture into the epidural space. However, the above method is still very difficult, since the complexity of tissues will still effect operation.

On the other hand, taking bone fracture surgery as an example, it will have a surgical incision on the skin for inserting a bone plate, so as to result in a wound with a large area, and the patient will be painful after the surgery. Recently, a minimally invasive surgery is an important technology for solving problems of the surgery mentioned above (that is, an open reduction internal fixation, ORIF surgery procedure), such as infection, poor and late wound healing. In details, the surgery can be performed through a few smaller wounds, so as to reduce the bleeding and the damage of tissue in the lesion zone. It also relieves pain to the patient. Moreover, the wound will be attractive after healing due to the smaller wounds.

However, in the limitation of vision, the minimally invasive surgery or the surgery for fixing bone plate needs assistances with medical imaging. Moreover, it also has problem to determine the position of screw hole of the bone plate when directly screwing a bone nail during the bone fracture surgery.

### SUMMARY

Accordingly, the present invention provides an ultrasound probe for accurately positioning on a target operation region. The ultrasound probe includes a body and an engaging element. The body contacts skin of a patient through one end thereof and can include an ultrasonic transducer and a groove. The groove can be designed at one side surface of the body and extends towards the end contacting the skin of the patient. The engaging element can be adjacent to the groove of the body for allowing an article to be engaged into or detached from the groove through the side surface of the body. In addition, the engaging element can be turned off and on between a first position and a second position with respect to the groove. When the engaging element turns off at the first position, the article can be engaged into the groove. When the engaging element turns on the second position, the article can be detached from the groove.

Preferably, the article can be a puncture needle. More preferably, the puncture needle can be a hollow structure for inserting a needle transducer to detect a distance between a tip of the needle transducer and the target operative region.

Preferably, the article can be a position needle for a bone nail.

Preferably, the body of the ultrasound probe can include a first ultrasonic transmission/reception region and a second ultrasonic transmission/reception region on two sides of the groove which be as a boundary. More preferably, one of the first ultrasonic transmission/reception region and a second ultrasonic transmission/reception region is capable of modulating a angle of an ultrasonic transmission/reception scanning.

Preferably, the scanning angle varies from 0 degree to 20 degrees. More preferably, the scanning angle varies from 5 degrees to 10 degrees.

Preferably, the engaging element is a rotatory switch or a latch.

### BRIEF DESCRIPTION OF THE DRAWINGS

The invention can be further understood by reading the following detailed description of the embodiment, with reference made to the accompanying drawings as follows:

FIG. 1 is a schematic view of an ultrasound probe according to the embodiment of the present invention;

FIG. 2 is a schematic view of an engaging element located at the first position with a puncture needle according to the embodiment of the present invention;

FIG. 3A and FIG. 3B are schematic views of the engaging element located at the second position with the puncture needle according to the embodiment of the present invention;

FIG. 4A, FIG. 4B, FIG. 4C and FIG. 4D are multi-angle scanning imaging results according to the embodiment of the present invention; and

FIG. 5 is a schematic view of using a positioning needle for a bone nail to confirm an operation position of a screw hole of a bone plate through a groove of a body according to another embodiment of the present invention.

### DETAILED DESCRIPTION

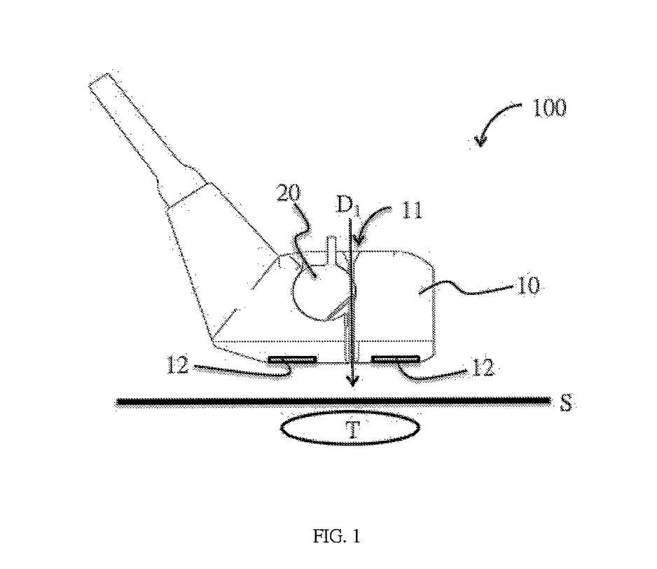

Accordingly, the present invention provides an ultrasound probe 100 as shown in FIG. 1 for accurately positioning on a target operation region T of a patient.

The ultrasound probe 100 includes a body 10 and an engaging element 20. The body 10 includes an ultrasonic transducer to contact skin S of a patient (not shown) by one end thereof. A groove 11 is designed at one side surface of the body 10, which is close to the end of the ultrasound probe 100. More preferably, the groove 11 is designed at a middle of the body 10 side surface, the present invention is not limited thereto. Moreover, the groove 11 can be a strip-like opening and extends from the end, which contacts the skin S of the patient, to have an extending direction D₁.

The ultrasound probe 100 further includes a first ultrasonic transmission/reception region 12 and a second ultrasonic transmission/reception region 13. The first ultrasonic transmission/reception region 12 and the second ultrasonic transmission/reception region 13 are sited at the end of body 10, which contacts the skin S of the patient, on two sides of the groove 11. One of the first ultrasonic transmission/reception region 12 and a second ultrasonic transmission/reception region 13 is capable of modulating a angle of an ultrasonic transmission/reception for performing a multi-angle scanning, so as to eliminate a visual blind zone. Preferably, the body 10 is a B-mode ultrasound transducer.

The engaging element 20 is adjacent to the groove 11 of the body 10 for allowing an article (not shown) to be engaged into or detached from the groove 11 through the side surface of the body 10. Preferably, the article can be but not limited to a puncture needle. Moreover, the engaging element 20 can be a rotatory switch or a latch.

Epidural anesthesia is taken as an example (that is, the article is the puncture needle) for illustrating the ultrasound probe 100 of the present invention and a method for operating thereof. First, the body 10 of the ultrasonic probe 100 is placed close to the skin S for obtaining a position of a target operative region T (that is, the epidural space) by at least one ultrasonic image and uses its center to align to the epidural space. Thus, two ultrasonic signal reflections of Ligamentum Flavum and Dura mater are presented at the middle of the ultrasonic image. Accordingly, a depth of the epidural space can be detected for an alignment of a puncture and planning a puncturing route.

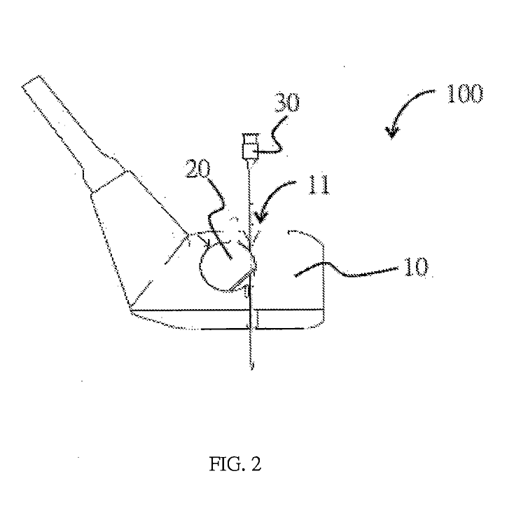

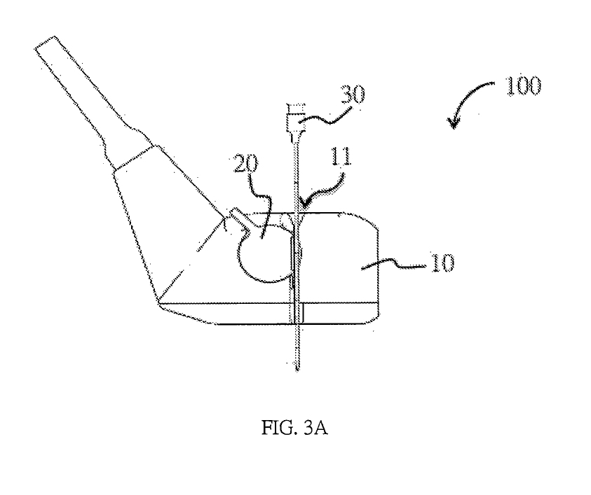

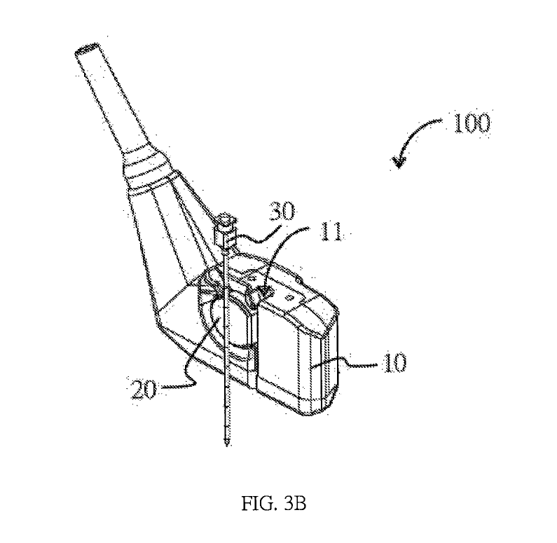

Please refer to FIG. 2, FIG. 3A and FIG. 3B. FIG. 2 is a schematic view of the engaging element 20 located at a first position with a puncture needle 30 according to the embodiment of the present invention. FIG. 3 and FIG. 3B are schematic views of the engaging element 20 located at a second position with the puncture needle 30 according to the embodiment of the present invention. In details, the engaging element 20 is located at the first position or the second position with respect to the groove 11. When the engaging element 20 is located at the first position with respect to the groove 11, the engaging element, that is, the rotatory switch, rotates towards the groove 11 so as to allow the puncture needle 30 to be engaged into the groove 11 as shown in FIG. 2. In particular, a distance between a tip of the puncture needle 30 and the target operative region T can be adjusted by adjusting the engaging element 20. Then, as shown in FIG. 3A, the engaging element 20 can be moved to be located at the second position when the tip of the puncture needle 30 arrives a predetermined depth. At that time, the puncture needle 30 can be laterally detached from the ultrasound probe 100 through the groove 11, disposed at the side surface of the body 10, as shown in FIG. 3B. Thus, a surgeon can use the puncture needle 30 alone for a preciously puncture in the following process.

In particular, the puncture needle 30 is a hollow structure for inserting a needle transducer. Thus, the puncture needle 30 can be detected from the distance above the epidural space 4-5 mm through the two ultrasonic signal reflections of Ligamentum Flavum(LF) and Dura mater(DM) so that doctors can adjust the force for adjusting the distance between the tip of the puncture needle 30 and the epidural space. Accordingly, the probability of piercing the epidural space can be reduced.

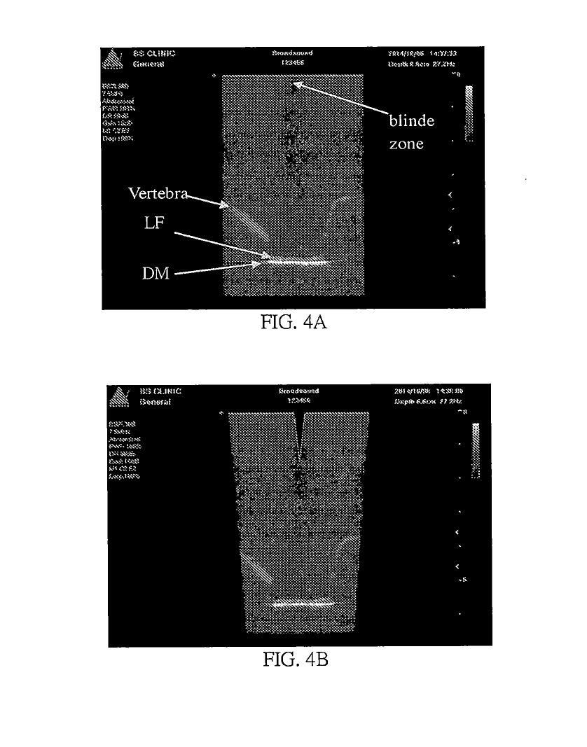

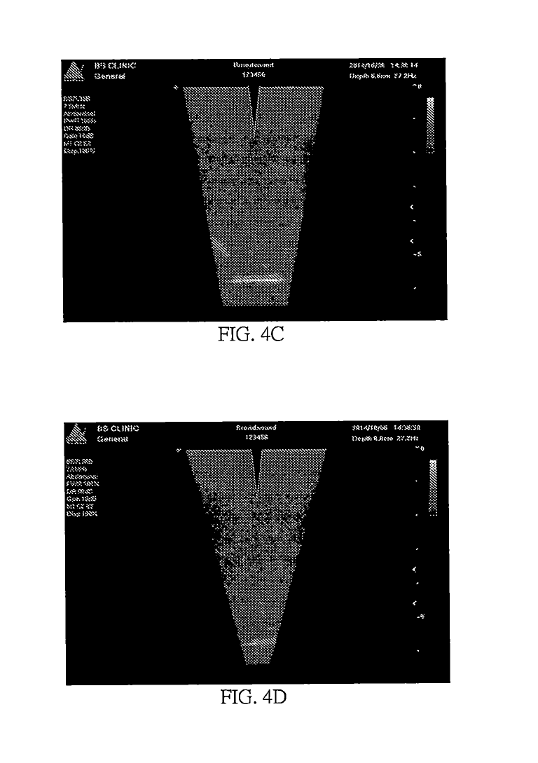

Because the ultrasound transducers are on two sides of the groove 11, it will form a blind zone on the image. Adjusting the scanning angle of the first ultrasonic transmission/reception region 12 or the second ultrasonic transmission/reception region 13 will combine a plurality of ultrasonic signals to define a size of the blind zone. Please refer from FIG. 4A to FIG. 4D, which are multi-angle scanning imaging results according to the embodiment of the present invention. In the embodiment of the present invention, the scanning angle of the first ultrasonic transmission region 12 and the second ultrasonic transmission region 13 have a plurality of ultrasonic emission points. Because emission timings of the ultrasonic emission points are different, the scanning angles of the first ultrasonic transmission/reception region 12 and the second ultrasonic transmission/reception region 13 can be adjusted so as to be combined on a focal plane with a depth. As shown in FIG. 4A, it is an ultrasonic image that is obtained when the scanning angles of the first ultrasonic transmission region 12 and the second ultrasonic transmission region 13 are adjusted to be at zero degree. As shown in FIG. 4B, it is an ultrasonic image that is obtained when the scanning angles of the first ultrasonic transmission region 12 and the second ultrasonic transmission region 13 are adjusted to be at 5 degrees. As shown in FIG. 4C, it is an ultrasonic image that is obtained when the scanning angles of the first ultrasonic transmission region 12 and the second ultrasonic transmission region 13 are adjusted to be 10 at degrees. As shown in FIG. 4D, it is an ultrasonic image that is obtained when the scanning angles of the first ultrasonic transmission region 12 and the second ultrasonic transmission region 13 are adjusted to be at 15 degrees. However, the present invention is not limited thereto. The larger the scanning angles of the first ultrasonic transmission/reception region 12 and the second ultrasonic transmission/reception region 13 are, the less the blind zone is. However, the depth of the focal plane will be affected. Preferably, the scanning angles are varied from 0 degree to 20 degrees, and the depth of the focal plane is suitable for performing an operation on subcutaneous tissues. More particularly, the scanning angles are varied from 5 degrees to 10 degrees for the epidural anesthesia.

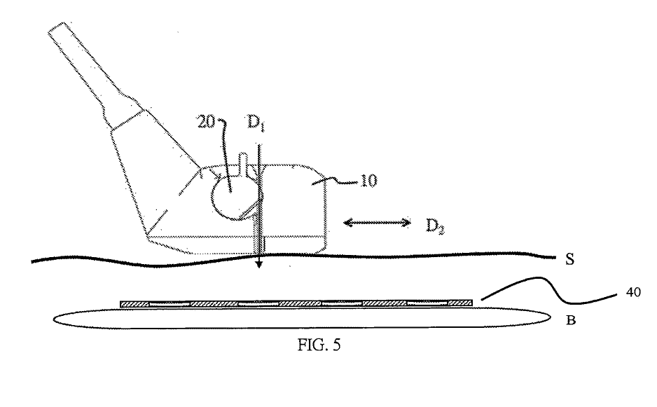

The ultrasound probe of the present invention can not only be applied on the epidural anesthesia but also can be applied on the surgery treatment of bone fracture. Please refer to FIG. 5, which is a schematic view of using a positioning needle for a bone nail to confirm an operation position of a screw hole of a bone plate 40 through a groove of a body according to another embodiment of the present invention. First, a damaged zone, such as the fracture or fragmentation, of bone is checked. The bone plate 40 is then inserted to be parallel to the bone B. The body 10 of the ultrasound probe 100 is attached to the skin S which is inserted with the bone plate 40, an ultrasonic scanning is performed along a direction D₂ to check at least one position of the screw hole of the bone plate 40. Then, the positioning needle for the bone nail is punctured along an extending direction D₁ of the groove for fixing a relative position between the bone plate 40 and the skin S. The bone nail can be punctured into the bone for reducing a wound area.

To sum up, the present invention provides the solution to the problem of spinal tissue puncture and assist the guiding of the puncture for improving the success rate of surgery and reducing the risk of failure. For the purposes of epidural puncture, it is difficult to plan a puncture path and provide an early alert and a real-time detection before the puncture needle arrives the target zone. Thus, the present invention provides an ultrasound probe is combined to an ultrasound scanning device and a real-time image display system for planning a puncture path through a multi-angle scanning. Moreover, the body of the ultrasound probe has the groove disposed on its side surface for inserting the puncture needle. Preferably, another ultrasonic transducer can be combined to perform a real-time function, and the puncture needle can be detached from the ultrasonic transducer laterally for reducing the interference of the ultrasound probe in the following operation.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

1. An ultrasound probe for accurately positioning on a target operative region of a patient, comprising:
a body contacting skin of a patient through one end thereof and comprising: an ultrasonic transducer; and a groove being designed at one side surface of the body and extending towards the end contacting the skin of the patient; and
an engaging element being adjacent to the groove of the body for allowing an article to be engaging into or detached from the groove through the side surface of the body; wherein the engaging element being turned off or on at a first position or at a second position with respect to the groove;
wherein the article is engaged into the groove when the engaging element is located at the first position; wherein the article is detached from the groove when the engaging element is located at the second position.

2. The ultrasound probe of claim 1, wherein the article is a puncture needle.

3. The ultrasound probe of claim 2, wherein the puncture needle is a hollow structure for inserting a needle transducer to detect a distance between a tip of the needle transducer and the target operative region.

4. The ultrasound probe of claim 1, wherein the article is a position needle for a bone nail

5. The ultrasound probe of claim 1, wherein the body comprises a first ultrasonic transmission/reception region and a second ultrasonic transmission/reception region on two sides of the groove.

6. The ultrasound probe of claim 5, wherein one of the first ultrasonic transmission/reception region and the second ultrasonic transmission/reception region is capable of modulating a angle of an ultrasonic transmission/reception scanning

7. The ultrasound probe of claim 6, wherein the scanning angle is varied from 0 degree to 20 degrees.

8. The ultrasound probe of claim 7, wherein the scanning angle is varied from 5 degrees to 10 degrees.

9. The ultrasound probe of claim 1, wherein the engaging element is a rotatory switch or a latch.
